(19) United States
(12) Patent Application Publication
(10) Pub. No.: US 2009/0182427 A1
(43) Pub. Date: Jul. 16, 2009

(54) VERTEBROPLASTY IMPLANT WITH ENHANCED INTERFACIAL SHEAR STRENGTH

(75) Inventors: Y. King Liu, Petaluma, CA (US); Jan R. Lau, Windsor, CA (US); Judson E. Threlkeld, Camas, WA (US); Michael T. Lyster, Riverwoods, IL (US); John Stalcup, Glen Ellen, CA (US)

Correspondence Address: KNOBBE MARTENS OLSON & BEAR LLP, 2040 MAIN STREET, FOURTEENTH FLOOR, IRVINE, CA, 92614, US

(73) Assignee: Osseon Therapeutics, Inc., Santa Rosa, CA (US)

(21) Appl. No.: 12/329,342

(22) Filed: Dec. 5, 2008

Related U.S. Application Data
(60) Provisional application No. 60/992,994, filed on Dec. 6, 2007.

(51) Int. Cl. A61F 2/28

(52) U.S. Cl. 623/16.11

(57) ABSTRACT

Methods and devices for augmenting bone, such as in performing vertebroplasty are disclosed. A bone implant with enhanced interfacial shear strength can include a container, such as a mesh bag, with a sidewall and an interior chamber portion. The sidewall can include an open-celled matrix and can be filled with a first media to promote bone ingrowth and enhanced interfacial shear strength. The interior chamber can be filled with a second media to prevent crack propagation. Delivery catheters with releasable coupling features to the implant are also disclosed.

The present application claims priority under 35 U.S.C. § 119(e) to U.S. Provisional Application No. 60/992,994 filed Dec. 6, 2007, which is hereby incorporated by reference in its entirety.

### SUMMARY OF THE INVENTION

In some embodiments, disclosed herein is a bone implant that includes a container, such as a mesh structure having a collapsed configuration and an expanded configuration. The mesh structure can have a sidewall and an interior chamber. The sidewall can have an open-cell matrix configuration. The implant can also include a first media configured to fill the sidewall of the mesh structure and promote bone ingrowth, and a second media configured to fill the interior chamber of the mesh structure and have a crack propagation arresting characteristic. The mesh structure can be at least partially bioresorbable in some embodiments. The first and/or the second media can include particles. The particulate density of the first media can be greater than, equal to, or less than the particulate density of the second media. The first media can includes particles in a concentration within the range of from about 50% to about 80% by weight in some embodiments. The second media can include particles within the range of from about 10% to about 50% by weight, or from about 25% to about 35% by weight. In some embodiments, the first media includes particles having a size within the range of from about 50 microns to about 500 microns, or within the range of from about 150 microns to about 300 microns. In some embodiments, at least one of the first and second media comprises PMMA. The sidewall of the bone implant can be porous, and have pores having a size, for example, of between about 0.5 mm and 1 mm, between about 1 mm and 2 mm, or more than 2 mm. In some embodiments, the mesh structure has a diameter of between about 1 mm and 4 mm in its expanded configuration.

Also disclosed herein is a kit for treating the spine, such as performing vertebroplasty. The kit can include a bone implant comprising a mesh structure having a collapsed configuration and an expanded configuration. The mesh structure can include a sidewall and an interior chamber. The sidewall can have an open-cell matrix configuration. The kit can also include a first media configured to fill the sidewall of the mesh structure and promote bone ingrowth, and a second media configured to fill the interior chamber of the mesh structure and have a crack propagation arresting characteristic. The kit can also include a vertebroplasty catheter comprising a proximal end, a distal, end, and an elongate tubular body, the tubular body having a central lumen extending therethrough. The mesh structure and the vertebroplasty catheter are configured to be releasably coupled together in some embodiments, such as when the mesh structure and the vertebroplasty catheter comprise complementary threaded attachment structures.

Also disclosed herein is a method for treating the spine, including the steps of: inserting an insertion device percutaneously into a vertebral body; introducing a bone implant comprising a mesh structure having a collapsed configuration and an expanded configuration, the mesh structure comprising a sidewall and an interior chamber, wherein the sidewall has an open-cell matrix configuration; filling the sidewall of the mesh structure with a first media having a bone ingrowth characteristic; and filling the interior chamber of the mesh structure with a second media having a crack propagation arresting characteristic. The method can also include the step of inserting a cavity-forming device through the insertion device into an area of cancellous bone in the vertebral body; and displacing cancellous bone with the cavity-forming device to create a cavity defined by a surface of cancellous bone.

Also disclosed herein is a method for treating the spine, including the steps of: inserting a deployment device into a vertebral body, the deployment device releasably carrying an inflatable container having a central cavity and a porous sidewall; inflating the container within the vertebral body; releasing the container within the vertebral body; and removing the deployment device from the vertebral body. The released container can include a first media within the pores of the sidewall and a second media within the cavity. The first media can be introduced into the pores prior to the inserting step, or following the inserting step in other embodiments. In some embodiments, the inflating step compacts adjacent cancellous bone. The method can also include the step of creating a cavity within the vertebral body prior to the inserting step. The pores can comprise spaces between fibers. In some embodiments, the pores can include open, interconnected cells in a porous matrix. The inserting step can be accomplished through an insertion cannula in some embodiments.

### BRIEF DESCRIPTION OF THE DRAWINGS

FIG. 1 is a side elevational cross sectional view of a vertebroplasty catheter releasably connected to a mesh bag, according to one embodiment of the invention.

FIG. 2 illustrates the vertebroplasty catheter-mesh bag system of FIG. 1 following proximal retraction of a stiffening wire.

FIG. 3 illustrates inflation of the mesh bag of FIGS. 1-2 following introduction of media.

FIG. 4 illustrates a threaded releasable coupling between catheter and mesh bag, according to one embodiment of the invention.

FIGS. 5-8 illustrate a method sequence for treating a vertebral body using a mesh bag and inflation media, according to one embodiment of the invention.

FIGS. 9-12 illustrate composites that can be used to fill the sidewall and interior chamber of a mesh bag, according to some embodiments of the invention. The mesh bag is not shown in these figures for simplicity. FIG. 9 illustrates a cross-section of two regions within the composite in place in a bone, according to one embodiment of the invention.

FIG. 10 further illustrates that a first region has a higher volume fraction of particles and a second region has a lower volume fraction of particles, according to one embodiment of the invention. In this illustration, the composite cement material substantially surrounds all of the particles and contacts the natural bone.

FIG. 11 illustrates the regions somewhat similarly to FIG. 10; however, this illustration shows that there are some particles which are only partially contacted or not contacted at all by the cement, according to one embodiment of the invention.

FIG. 12 is a cross-section that illustrates a composite having a gradient of local volume fraction of particles, according to one embodiment of the invention.

### DETAILED DESCRIPTION

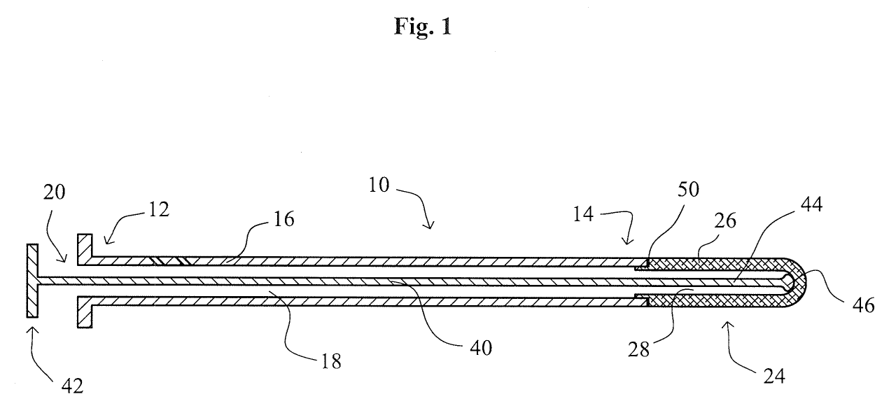

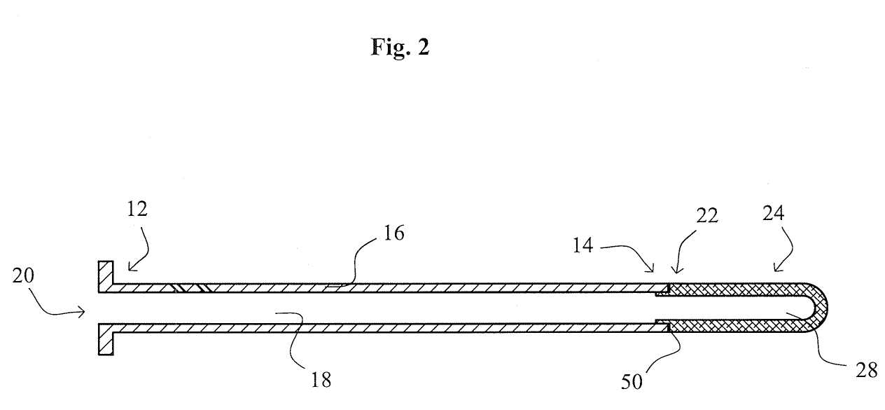

Referring to FIGS. 1 and 2, there is illustrated a side elevational cross sectional view of a vertebroplasty catheter 10, in accordance with one aspect of the present invention. Vertebroplasty catheter 10 is adapted for implanting a two-part bone cement system such as that disclosed in U.S. patent Ser. No. 11/626,336, filed Jan. 23, 2007, entitled Bone Cement Composite Containing Particles in Non-Uniform Spatial Distribution and Devices for Implementation, to Y. King Liu et al, U.S. Publication No. 2007/0185231 A1, the disclosure of which is incorporated in its entirety by reference herein. In general, a bolus of vertebroplasty cement is injected such that an outer layer comprises an enhanced bone ingrowth characteristic such as by having a higher particulate density, and an inner core is provided having an enhanced crack propagation resisting characteristic, such as by including a lower particulate density.

The vertebroplasty catheter 10 comprises a proximal end 12, a distal end 14, and an elongate tubular body 16. Tubular body 16 is provided with a central lumen 18, extending between a proximal port 20 and a distal port 22. Tubular body 16 may comprise any of a variety of materials known in the orthopedic catheter arts, such as stainless steel, Nitinol, or rigid polymers including PEEK, PEBAX, high density polyethylene, and others known in the art. Tubular body 16 may comprise any of a variety of lengths, depending upon the desired access site and treatment site. In general, for vertebroplasty applications, the length of tubular body 16 will be within the range of from about 7 cm to about 35 cm. The outside diameter of tubular body 16 would generally be less than 12 mm, in certain embodiments less than about 9 mm, and, optimally, less than about 7 mm. Further non-limiting examples of vertebroplasty catheters and other delivery system elements that can be used or adapted for use herein can be found, for example, in FIGS. 1-18B and paragraphs to [0137] of U.S. patent application Ser. No. 12/029,428 to Liu et al., filed Feb. 11, 2008, and hereby incorporated by reference in its entirety.

A collapsible inflatable mesh bag 24 is releasably carried by the distal end 14 of the catheter 10. The collapsible inflatable mesh bag 24 is adapted for inflation and deployment at a treatment site as is discussed below. Mesh bag 24 may be from about 1 cm to about 4 cm in inflated diameter, and may be approximately spherical in shape. However, other elliptical, elongate, or irregular shapes may be used, depending upon the desired clinical result. The mesh bag 24 is pliable and malleable before its interior space 28 is filled with the contents described elsewhere herein. The mesh bag 24 may be constructed of any of a variety of woven or nonwoven filaments, which may be woven, knitted, braided or otherwise configured having a filament density that will allow ingress and egress of fluids, blood vessels and fibrous tissue as well as bony trabeculae to facilitate bone growth therethrough. Sponge like materials may also be used such as flexible polymers having an open cell tortuous pathway matrix for retaining the powder or bone cement formulation disclosed elsewhere herein. Optionally, the bag may be substantially water tight prior to inflation.

Preferably, the effective pore size through the mesh bag 24 will be large enough to facilitate bone growth therethrough, but small enough to inhibit or to slow the passage of bone cement utilized to inflate the bag. The pore size and pore density is controllable in the manufacturing process, as is known in the art. For example, if polymethylmethacraylate (PMMA) bone cement is desired for the inflation medium, the pore size is preferably less than about 0.5 mm to about 1.0 mm. Alternatively, if bone graft or biocompatible ceramic granules are utilized as inflation media, pore sizes of about 1.0 mm or greater may be used.

The collapsible mesh bag 24 may be formed from any of a variety of materials suitable for medical implantation. In general, these include permanent implantable materials, and bioabsorbable materials. Permanent implantable materials include metal wire filaments, such as stainless steel, titanium, Nitinol, or others known in the art. Permanent implantable polymeric filaments include nylon, PTFE, high density polyethylene, PEBAX, PEEK, and others known in the art. Suitable bioabsorbable filaments may comprise any of a variety of known polymers such as polylactides (PLA), polyglycolic acids, and analogs and derivatives thereof. A variety of materials suitable for implantation and formation into filaments are disclosed in U.S. Pat. No. 5,545,208 to Wolff (e.g., at FIGS. 1-5 and 8-17 and the corresponding disclosure at col. 7 line 50 to col. 9 line 34) and U.S. Application Publication No. 2006/0129222 to Stinson (e.g., at FIG. 1-3 and 12-18 and paragraphs [0048] to [0113]); the disclosures of which are incorporated in their entireties herein by reference.

Preferably, the wire or polymeric filaments utilized to form the collapsible inflatable mesh bag 24 are configured to maximize the proliferation of cells and vasculature through the apertures to facilitate bone ingrowth. Generally, greater proliferation of mesenchymal cells and vasculature through the apertures of the membrane into the bone defect area, yields greater healing potential of the body. Apertures that are too small do not optimize the proliferation of cells in the vasculature through the apertures of the membrane. Apertures ranging from about 1,000 microns to about 3,000 microns are often utilized, and, in certain embodiments, from about 1,500 microns to about 3,000 microns. Greater aperture sizes may also be used, depending upon the desired viscosity and inflation pressure for the second media. Preferably, the result is an optimized environment for osteoconduction, which involves the incorporation of a three-dimensional interconnected porous framework to conduct the ingrowth of new living bone through the mesh bag 24. Osteoconduction may be facilitated or enhanced by the addition of any of a variety of additives, such as mesenchymal stem cell and/or bone marrow aspirates. Platelet rich plasma, growth factors, peptides and/or proteins, and/or any of a variety of synthetic or natural osteoinductive or osteogenic materials may also be added to the collapsible inflatable mesh bag 24.

As described in detail in U.S. Publication No. 2007/0185231 A1 to Liu et al., one aspect of the improved vertebroplasty of the present invention is the provision of a bone cement bolus having an outer shell with an enhanced bone ingrowth characteristic, filled by an inner core with an enhanced crack propagation arresting characteristic. The bone ingrowth characteristic is provided by implanting and inflating the collapsible inflatable mesh bag 24 within the cancellous bone of a vertebral body. The collapsible inflatable mesh bag 24 is provided with a tortuous, open cell interconnected porous configuration, such as a sponge, or microporous structure which facilitates bony ingrowth. The mesh bag 24 may be implanted following impregnation with an agent or media such as a bone ingrowth facilitating, higher particulate density bone cement as discussed in the Liu et al. publication. The mesh thus acts as a scaffold or support structure to support a substantially uniform layer of bone cement or other material that is intended for implantation as a liner or shell around a core as will be discussed. The mesh bag 24 is subsequently inflated by the second bone cement formulation discussed in Liu et al., which is optimized to enhance the crack propagation arresting characteristic. In general, the inflation media for the catheter 10 of some embodiments will have a lower particulate density than the particulate density impregnated within the wall of the collapsible inflatable mesh bag 24.

Referring to FIG. 1, the collapsible inflatable mesh bag 24 comprises a side wall 26, and an interior chamber 28. Interior chamber 28 is placed in communication with proximal port 20 by way of central lumen 18. In some embodiments, interior chamber 28 can be divided into multiple compartments, such as with baffles. The side wall 26 may have a thickness that corresponds to the desired thickness of the bone growth layer. In general, thicknesses in the range of from about 1 mm to about 3 mm are sufficient as was shown by Hulbert et al. that bony ingrowth beyond 2 mm did not increase the interfacial shear strength if the bony ingrowths into the interconnected pores created by the simultaneous osteoclastic and osteoblastic activity as was shown by Dai et al.

An optional stiffening wire 40 extends throughout the length of the central lumen 18, from a proximal end 42 to a distal end 44. Distal end 44 is provided with a distal tip 46, having a blunt or atraumatic distal end, such that it may press against the interior of the collapsible inflatable mesh bag 24 without risk of perforation. Stiffening wire 40 may be positioned within tubular body 16 as illustrated in FIG. 1 for the purpose of providing beam-column strength to the mesh bag 24 as it is advanced to the treatment site.

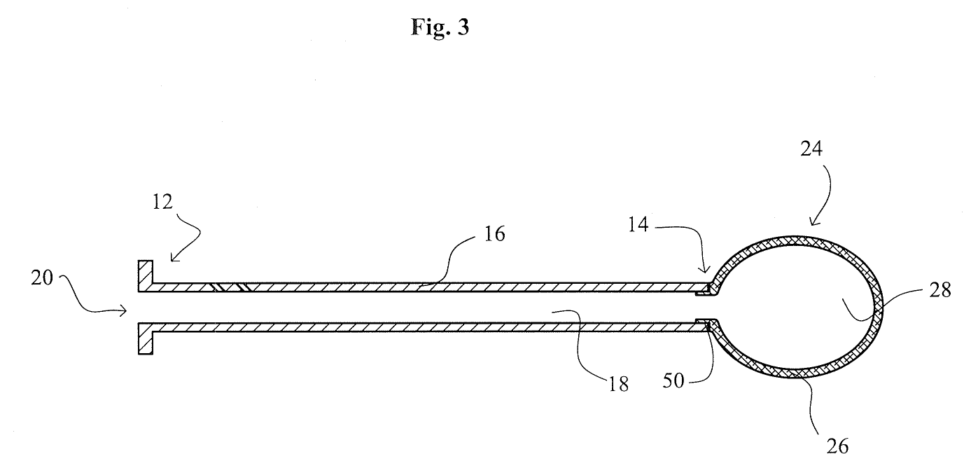

Following placement of the collapsed mesh bag 24 at the treatment site, the stiffening wire 40 is proximally retracted from the central lumen 18 to produce the construct illustrated in FIG. 2. At that point, bone cement, such as the part B bone cement optimize for crack propagation arresting characteristics, and generally having a lower particulate density than the part A bone cement impregnated within the mesh bag 24, is introduced through central lumen 18 and into mesh bag 24. The mesh bag 24 is thereby inflated by the introduction of bone cement, to produce the inflated configuration such as that illustrated in FIG. 3. The mesh bag both delivers a uniform layer of bone cement formulation to the interface with cancellous bone, as well as inhibits extravasation or leaks which could otherwise occur due to the inflation pressure.

The illustrations in FIGS. 1 through 3 are simplified to illustrate the basic principles of some embodiments of the invention, with other features removed for simplicity. For example, an outer tubular introduction sheath may be provided, through which the catheter 10 is axially distally advanced to the treatment site. Following positioning of the mesh bag 24 at the treatment site, the outer tubular sleeve may be proximally retracted to expose the mesh bag 24, prior to its inflation followed by introduction of the part B bone cement.

A releasable coupling 50 is provided between the collapsible mesh bag 24 and the tubular body 16. Releasable coupling 50 enables inflation of the mesh bag 24 at the treatment site thereafter enabling uncoupling and removal of tubular body 16 leaving the mesh bag 24 in place at the treatment site. Any of a variety of complementary engagement and release structures may be utilized, for releasable coupling of the mesh bag 24 to the tubular body 16.

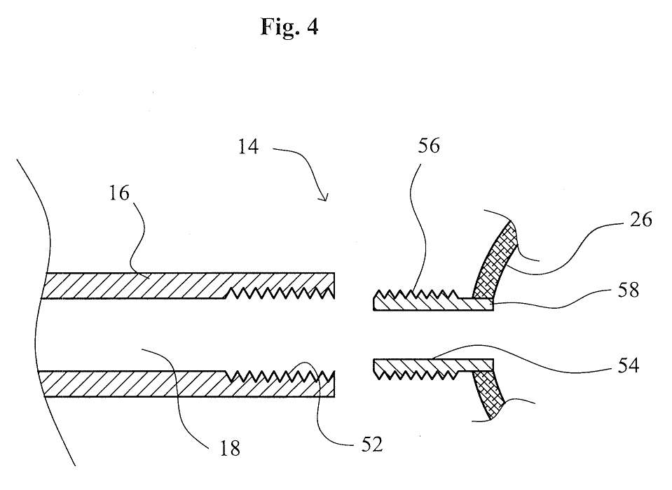

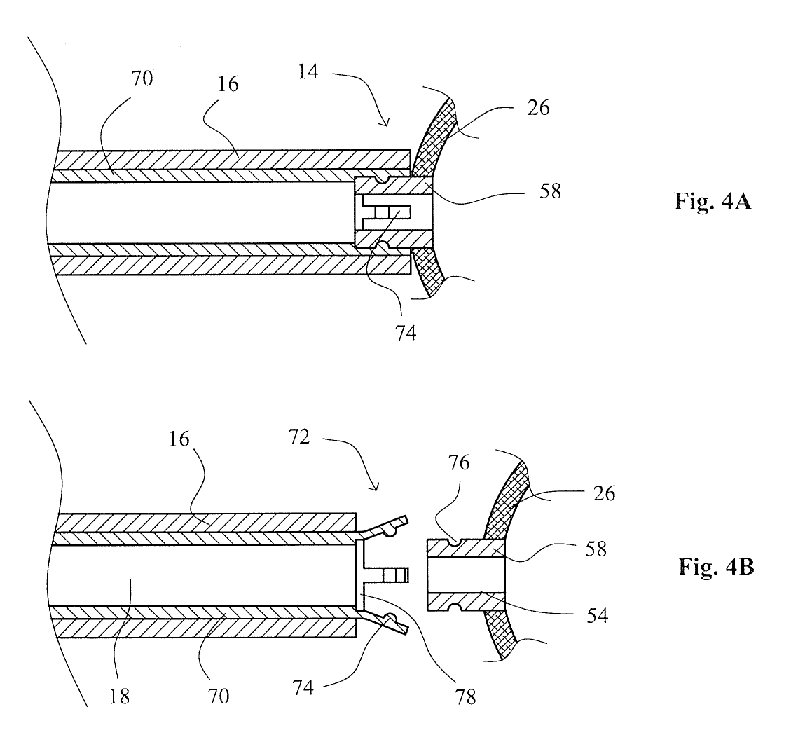

Referring to FIG. 4, a simple threaded engagement is illustrated. The distal end 14 of tubular body 16 is provided with a threaded attachment structure such as a female thread 52. The mesh bag 24 is provided with a connector 54 having a male thread 56 adapted for threadable engagement with the female thread 52 on tubular body 16. Connector 56 may be secured to the mesh bag 24 in any of a variety of ways, depending upon the materials of the mesh bag 24. For example, for mesh bag 24 comprising a stainless steel woven wire filament, the mesh bag 24 may be soldered, brazed, or otherwise secured at an attachment point 58 to the connector 56.

Referring to FIGS. 4A-4B, one embodiment of an alternate engagement mechanism is disclosed. The distal end 14 of tubular body 16 is provided with an inner catheter member 70 that is axially movable with respect to outer catheter member 16. Distal end 72 of inner catheter member 70 can be a flange with a protrusion 74 configured to releasably engage a complementary groove 76 on a connector 54 on the mesh bag 24. The flange 72 can have, for example, a hinge or pivot point, or a radially outward bias that promotes detachment when inner catheter 70 is no longer axially constrained within outer catheter 16 as illustrated in FIG. 4B. The inner catheter 70 including distal end 72 can be made, in some embodiments, out of a shape memory material such as, e.g., nitinol or eligloy, and heat set to the appropriate shape, such as shown. Distal end 72 of inner catheter member 70 also includes a nozzle 78 to control flow of bone cement or other media into corresponding connector 54 of the mesh bag 24. Alternatively, any of a variety of moveable interference structures may be utilized to temporarily retain mesh bag 24 in engagement with tubular body 16.

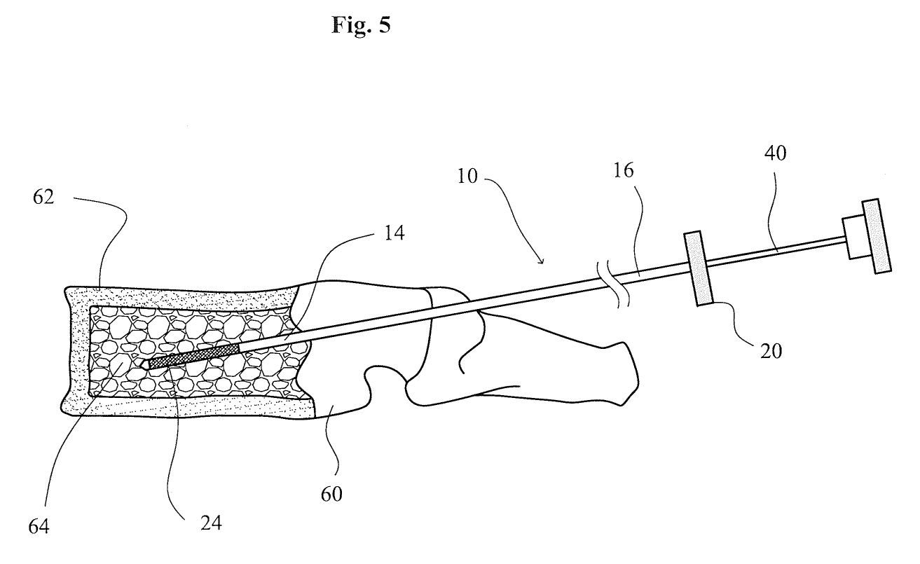

Referring to FIG. 5, there is illustrated a side elevational view of a vertebral body 60. Vertebral body 60 comprises an outer cortical bone shell 62, containing an inner cancellous bone 64. The catheter 10 is in position with the distal end 14 advanced through a predrilled tract into the cancellous bone 64. The mesh bag 24 is positioned at the desired treatment site within the vertebral body 60. Stiffening wire 40 is in position within the catheter 10, to maintain the mesh bag 24 in its low profile configuration for introduction to the treatment site.

In some embodiments, a cavity can be formed prior to mesh bag insertion, such as in the performance of kyphoplasty. Cavity-creating catheters and methods that can be used prior to mesh bag 24 insertion are described, for example, in paragraphs [0107] to [0016] and FIGS. 16A-17C of U.S. patent application Ser. No. 12/029,428 to Liu et al., previously incorporated by reference in its entirety.

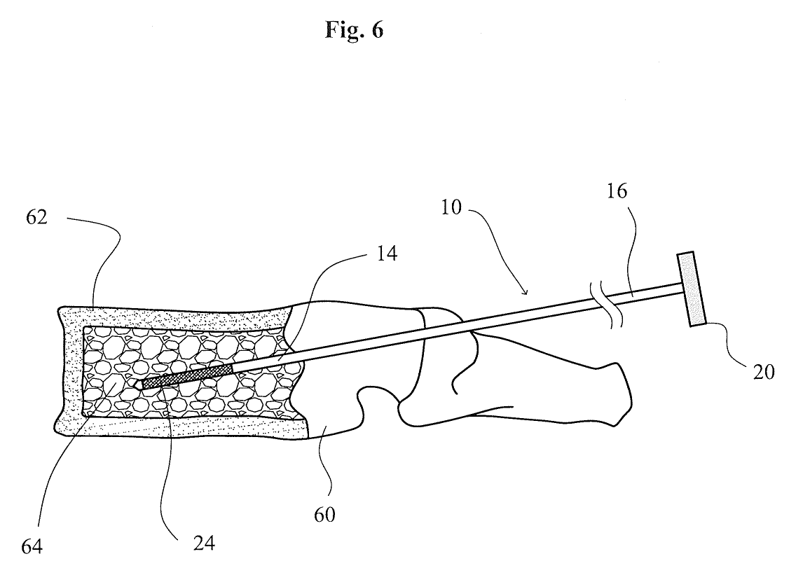

Referring to FIG. 6, the stiffening wire 40 has been proximally retracted from the catheter 10, leaving the mesh bag 24 in position at the treatment site, and in communication with the proximal port 20 by way of central lumen 18.

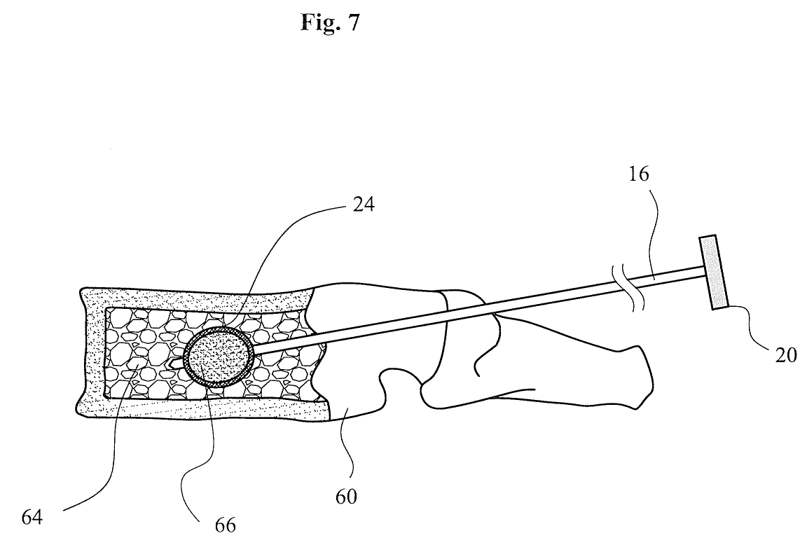

Referring to FIG. 7, the mesh bag 24 is illustrated in its inflated configuration. This is accomplished by coupling a source of inflation media 66 to the proximal port 20 on catheter 10, and introducing inflation media into the mesh bag 24 by way of central lumen 18. Preferably, the inflation media comprises bone cement such as PMMA, and, in particular, bone cement formulated as mixture B described above having a relatively lower particulate density than the part A cement impregnated within the wall of the mesh bag 24.

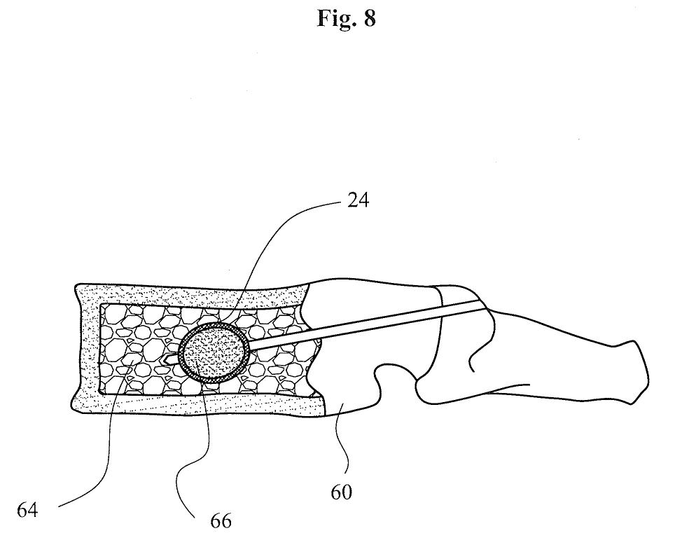

Referring to FIG. 8, the catheter 10 has been uncoupled from the mesh bag 24, such as by rotation of the tubular body 16 with respect to the mesh bag 24 in the case of a threaded engagement such as that illustrated in FIG. 4. Alternatively, any of a variety of release mechanisms may be activated, depending upon the releasable connection between the catheter 10 and mesh bag 24.

The resulting construct is a first bolus of bone cement having an outer layer of a part A formulation supported by and infiltrated through the sidewall 26, e.g., tortuous open pore structure of mesh bag 24, enclosing an inner core of a second bolus of a part B bone cement formulation within the interior chamber 28 of the mesh bag 24, as has been discussed elsewhere herein. In the event of a mesh bag 24 made from a stainless steel or other wire filament, mesh bag 24 will be permanently incorporated into the vertebral body. In an embodiment where the mesh bag 24 comprises an absorbable filament weave or fabric, the filament will gradually absorb over time, and the resulting pores will be filled by bone growth.

### MEDIA EXAMPLES

Non-limiting examples of media, such as bone cement, that can be used to fill an expandable container such as mesh bag 24 such as described above are described, for example, in FIGS. 2-5 and paragraphs [0079] to [0105] of U.S. Pat. Pub. No. 2007/0185231 A1 to Liu et al., incorporated by reference herein in its entirety. As noted, in some embodiments, a first media can be utilized to fill the sidewall 26 of the mesh bag 24 to promote bone ingrowth and osteoconduction, while a second media can be utilized to fill the interior chamber 28 of the mesh bag to prevent crack propagation. In the figures below and some of the description, the mesh bag 24 has been omitted for brevity; however, it will be understood that the composites described below can be used to fill a container such as described above. Some examples of bone ingrowth and crack-arresting media are described below; media having other advantageous features can also be utilized as well.

The composition regime of a composite exemplified by an optimal weight fraction of particles (which is greater than about 25%, and less than about 35% in a preferred embodiment) in the cement can be described as a crack-arresting regime. Based on the known proportion of the particles and the polymer, it can be expected that such a composite contains a substantially continuous phase of hardened acrylic cement that surrounds particles, which infrequently touch each other. It is believed that cracks, which originate in the substantially continuous polymeric phase, are only able to propagate for a short distance before they reach the hole of a particle, which then arrests the growth of the crack propagation. If a composite contains an even higher volume fraction of particles, it can exhibit another regime of behavior in vivo. In such a situation, there would again be at least some of a continuously interconnected phase of hardened acrylic cement, but, at the same time, many of the particles would have direct contact with one or more adjacent particles. If the particles are bioresorbable, resorption of the particles and ingrowth of new bone may occur simultaneously and could be expected to eventually leave ingrowth of natural bone into the bone cement. The situation in which bone has grown about 2 mm or more into the polymeric phase can be expected to yield especially good interfacial shear strength. This situation can be referred to as the “bone ingrowth” regime.

The invention provides an improved bone cement composite, whose bulk provides the fatigue life typically attainable with particle-containing cement, and also which further exhibits a greater interfacial shear strength at the bone-cement interface than would normally be obtained using pure bone cement.

The bone cement composite is believed to operate in the crack arresting regime throughout most of its bulk and in the bone ingrowth regime near the interface with natural bone (e.g., within the sidewall 26 of mesh bag 24). The enhanced shear strength at the bone-cement interface may allow many more implants to last the lifetime of the patient without ever needing revision surgery.

In general, the foregoing is achieved in some embodiments by providing a bone cement composite in which the local volume fraction of particles in the composite is spatially non-uniform in a controlled manner.

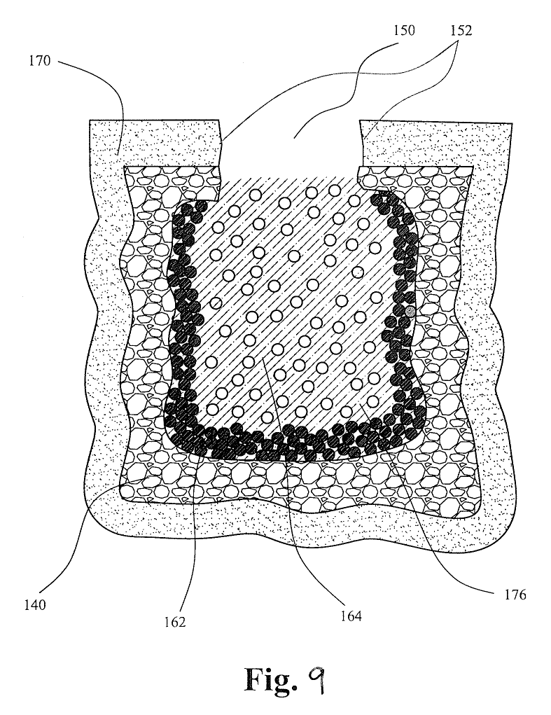

One embodiment of the invention is the configuration of the composite as it exists in the body of a patient after completion of the surgical procedure, in which the composite has two regions. This is illustrated in FIG. 9. FIG. 9 illustrates a portion of a bone 140, which has a surgical site 150. The surgical site 150 may have a potential cavity opening 152, and the potential cavity may contain composite 160. Composite 160 may comprise first region 162 and second region 164, which differ from each other in some respect. In FIG. 2, region 162 generally adjoins the bone 140, which defines the boundary of potential cavity opening 152. In some embodiments, region 164 may be generally surrounded by region 162 and generally may be free from contact with bone 140 which forms the boundary of potential cavity opening 152. In some embodiments, localized exceptions or anomalies can be present as well.

The invention will be described primarily herein in the context of introducing the composite bone cement as described herein into a vertebral body. However, it is contemplated that the composites disclosed herein can be introduced into a wide variety of bones throughout the body, and optionally in conjunction with the prior or concurrent formation of a cavity. Such bones may include, for example, the pelvis, the femur, the fibula, the tibia, humerus, ulna, radius, ribs, or various component structures of the cranial or facial skull. A wide variety of applications for this method can include therapeutic intervention for degenerative, infiltrative, traumatic and/or malignant defects of bone that include but are not limited to: Paget's disease, osteoporosis, osteomalacia, myeloma, metastatic epithelial malignancies, primary or metastatic sarcomas, osteogenesis imperfecta, osteochondromas and/or other non-metastatic deformative defects of bone including hemangiomas.

In addition, the invention can be described in the context of introduction of the composite into a vertebral body to restore vertebral body height, or minimize further degeneration of the vertebral body. In addition to filling a cavity in a bone, a composite may be utilized in any of a variety of other applications in which adhesion of a bone or non-bone prosthesis or device to a bone is desirable. For example, the composite may be utilized to assist in the fixation of any of a variety of devices to an interior or exterior surface of a bone, such as, for example, fixation of a medullary nail or rod, screws, plates, and other stabilization, fixation or mobility preservation hardware. Specific applications can include fixation of a total shoulder or total hip replacement, such as by fixation of a prosthesis stem within a medullary canal. The composite can also be used for reconstructive applications, e.g., reconstruction of congenital abnormalities, posttraumatic reconstruction of facial structures, pelvic and/or other bony sites, or postresection reconstruction in patients with epithelial or bony malignancies, including, but not limited to, head and neck carcinomas, pelvic sarcomas or discrete bone metastases following resection or other ablative procedures including radiofrequency (RF) and high intensity focused ultrasound (HIFU) ablation therapies.

The composite disclosed herein can additionally be utilized to assist in the attachment of any of a variety of bone anchors, suspension slings, or implantable diagnostic or therapeutic devices to bone, as will be apparent to those skilled in the art in view of the disclosure herein. Further, the composite may additionally be utilized to stabilize or secure a bone graft, allograft, synthetic bone grafts, or other implants within or adjacent a bone.

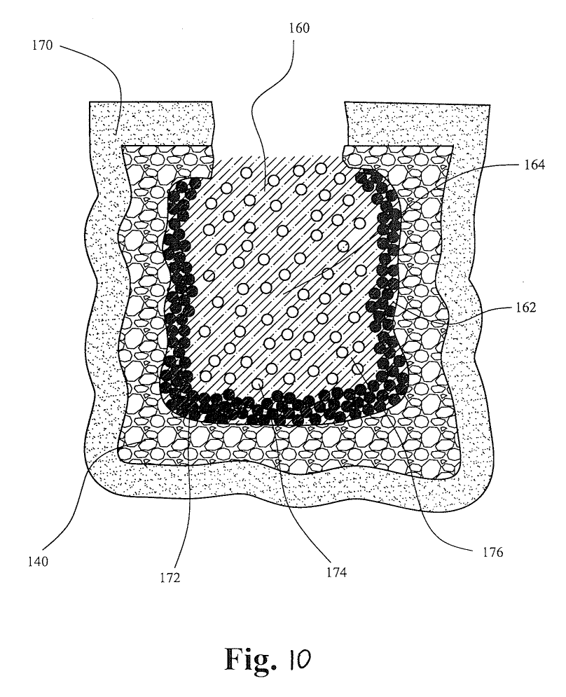

Regions 162 and 164 are illustrated in more detail in FIG. 10 by further showing that regions 162 and 164 may further contain, respectively, particles 172 and 174. In FIG. 10, as well as in other similar figures herein, the particles 172 and 174 are illustrated as being spheres of equal diameter. However, it is to be understood that this is only an idealization for ease of illustration, and in reality any of the particles 172, 174 could vary in any one or more of the following attributes, such as, for example, size, shape, size distribution, shape distribution, or other geometric characteristics. Particles 172 and 174 could be either identical to each other or different from each other in some respect, as discussed elsewhere herein.

In describing the presence of particles in cement, the term concentration (of particles) is used herein as a generic term referring to either volume fraction or weight fraction of particles in the cement. If a concentration of particles is reported as a weight fraction, as would be understood by one of ordinary skill in the art, a corresponding volume fraction of particles can be calculated if the mass densities of the particle material and the mass density of the cement are known. If the mass density of the particle material and the mass density of the cement happen to be identical, then the weight fraction and the volume fraction of the particles would be numerically identical. If the two mass densities are unequal, then numerical calculations can convert from mass fraction to volume fraction or vice versa, as known by those with skill in the art.

In some embodiments, at least some of the composite 160 can contain a continuous phase of cement 176, which may have dispersed solid particles 172 and 174 within the cement. In both region 162 and region 164, the composite could have a non-zero local volume fraction of the particles 172 and 174. The non-zero local volume fraction of particles 172 and 174 may be such that the composite has fatigue life which is longer than the fatigue life of particle-free or substantially particle-free cement. Within the composite, the local concentration of the particles 172 in region 162 may be different from the local concentration of the particles 174 in region 164. As illustrated in FIGS. 9 and 10, region 162, adjoining bone (e.g., within the sidewall 26 of mesh bag 24), could have a greater non-zero local volume fraction of particles 172, and region 164, generally not adjoining bone (e.g, within interior cavity 28 of mesh bag 24), may have a lesser non-zero local volume fraction of particles 174.

In the region designated 164, away from the immediate vicinity of the bone-composite interface, the concentration of particles 174 may be described by a weight fraction designated a. For example, this concentration of particles 174 may be in the range of approximately at least about 10% by weight to approximately no more than about 50% by weight. In another embodiment, the concentration of the particles 174 in region 164 may be in the range of approximately at least about 20% by weight to approximately no more than about 40% by weight. In yet another embodiment, the concentration of particles 174 in region 164 are preferably at least about 25% by weight but no more than about 35% by weight. In another embodiment, the concentration of particles 174 in region 164 is preferably about 30% by weight. The concentration of the particles 174 may be selected, at least in part, so as to provide desired fatigue properties of the resulting composite. Although about 30% weight concentration of particles has been reported in the literature to be the optimum concentration for the reported combination of materials, more generally, the particle concentration which produces the best fatigue properties may be unique to particular combinations of particle composition and properties and cement composition and properties. In region 164 of the composite, the properties of the composite may be such that the weight-bearing behavior can be described as being in the “crack arresting” regime. In this regime, generally speaking, most of the particles 174 may be immediately surrounded by cement 176, without being in direct contact with other particles 174. In FIG. 2, region 164 is illustrated as containing particles 174, in which at least most of the particles 174 do not touch any other particle 174. On average, such particles 174 may be separated from each other by only a small number of particle diameters or even by just a fraction of a particle diameter. This situation means that, on average, such a distance is the greatest length to which a crack in cement 176 is likely to grow before encountering a particle 174 which would arrest the growth of the crack propagation. Once the crack is arrested, additional cyclic loading may be needed to either initiate new crack(s) or propagate existing crack(s). This is believed to be the primary mechanism by which the presence of particles such as particles 174 can improve fatigue properties in this regime. However, other mechanisms may contribute to enhance the fatigue life in this regime as well.

In FIG. 9, the more densely packed region 162 is illustrated as containing particles 172, in which most of particles 172 directly touch other particles 172. At the same time, particles 172 may be at least partially surrounded by cement 176. In the immediate vicinity of the bone-cement interface, in region 162, the cement composite may have a local volume fraction of particles 172 which is designated by P. It can be noted that, based on geometric packing considerations and with assumption of spherical equally-sized particles, the maximum possible volume fraction of particles under any circumstance is no more than about 70%, with some variation possible depending on exact packing arrangement of particles and with the possibility that if there are multiple sizes of spherical particles or if there are non-spherical particle shapes, the number could be somewhat higher than about 70%. As discussed elsewhere herein, with knowledge of the respective mass densities of the particles and the cement, a relationship could be calculated by one of skill in the art between local volume fraction of particles and the local mass fraction of particles. In region 162, which is in the immediate vicinity of the bone-cement interface, the concentration of particles 172 may be in the range of about 50% to about 80% by weight, or more preferably about 60% to about 80% by weight. In such an embodiment, a significant fraction, such as more than about 50% of the particles 172 in region 162 of the composite, may have direct contact with a nearby particle 172. In other words, a particle 172 which is directly in contact with bone, could biodegrade, and replaced by new bone. Then, the bone can further come in contact with another particle 172 which had been in contact with the earlier-existing particle 172 before that particle was replaced by bone. Upon this occurrence, there may be a repetition of the method of particle resorption and bone ingrowth. By this method, ingrowth of a continuously connected network of natural bone into the composite may proceed for a distance of some number of particle diameters into the composite. For this reason, such a composite may be referred to as being in the “bone ingrowth regime.”

In general, in the immediate vicinity of the bone-cement interface (region 162), the composite may have a local volume fraction of particles 172 that is larger than the local volume fraction of particles 174 away from the immediate vicinity of the bone-cement interface (region 164). Region 162 may have a local volume fraction of particles that touch others, which puts it in the bone ingrowth regime. This is believed to help improve the strength of the bone-cement bond, such as the interfacial strength in shear. This is because shear strength is provided by bone ingrowth, and the amount of the ingrowth can be expected to increase with the concentration or volume fraction of the particles and the degree to which the particles contact each other to form inter-touching particles (which can be expected to increase with the local concentration or volume fraction of the particles). A region of composite having a relatively high local concentration of particles can be expected to contain a substantial number of particles 172, which directly touch other particles 172. The presence of particles 172, which directly touch other particles 172, can be expected to create interconnected particles, which in turn can be expected to help produce bone ingrowth by bone resorption and ingrowth. Again, however, it is not wished to be restricted to any of these theories or explanations.

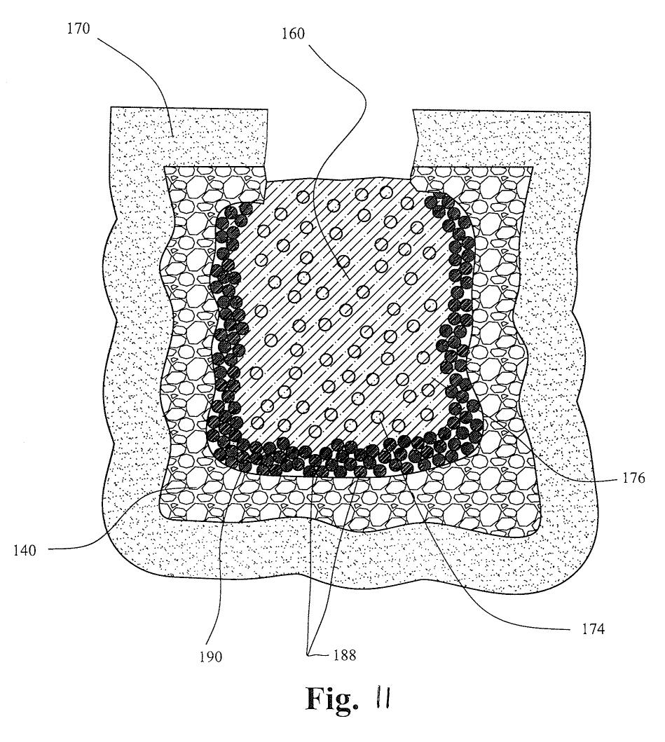

The immediate vicinity of the bone-composite interface can be defined herein to mean a distance of somewhere in the range of approximately 0.1 mm to approximately 2 mm, or no more than about 2 mm. Also, a local particle concentration can be defined as the weight or volume (depending on whether volume fraction or weight fraction is being discussed) of particles contained in a space, divided by the total weight or volume of all material contained in that space, wherein the space is at least approximately equiaxial in all three orthogonal dimensions and has a volume which is sufficient to contain at least approximately 3 particles or fractions of particles. For present applications, a typical average overall dimension or diameter of the particles 162 and 172 may be at least about 50 micrometers to no more than about 500 micrometers in some embodiments, and at least about 150 micrometers to no more than about 300 micrometers in another embodiment. In FIG. 10, the particles 174 are illustrated as being completely surrounded by cement. However, this is not essential and another embodiment of the invention can include particles 174 which are less than completely surrounded by cement. In FIG. 11, particles 188 are in only partial contact with cement. Furthermore, there may be particles such as particles 190, which are not in contact with any cement.

It is further possible, in still another embodiment of the invention, that even though most of the bone-composite interface occurs with the bone 140 contacting region 162 as illustrated in FIG. 9, there might be some isolated places where such an identifiably different region 162 does not separate region 164 from cancellous bone 140, and, for example, the region 164, operating in the crack-arresting regime, might contact cancellous bone 140. The outer layer of cortical bone 170 is also shown.

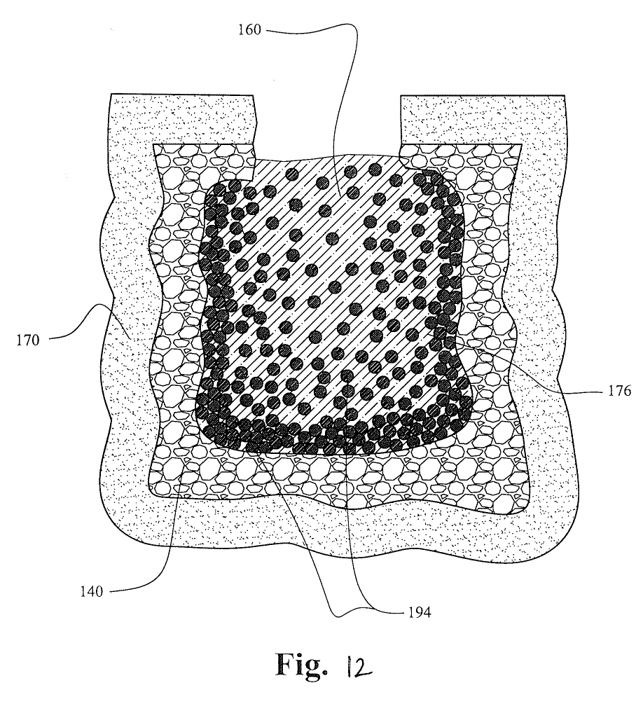

Description, for example of FIGS. 9-11 herein refers to a composite which contains identifiable regions such as regions 162 and 164 within the composite. Alternatively, as yet another embodiment of the invention, it is possible that the local volume fraction of particles may exhibit spatial non-uniformity, but without always having sharply-defined identifiable regions 162 and 164 as already illustrated. For example, there may be a gradient of local volume fraction of particles from one place to another within the composite. This is illustrated in FIG. 12. In FIG. 12, the particles 194 closest to the bone generally touch other particles, and the particles 194 in the interior of the composite generally do not directly touch other particles, but the variation between these two situations is more gradual than was illustrated in FIGS. 10 and 11. In FIG. 12, particles 194 generally represent the same particles as particles 172 and 174 in FIGS. 10 and 11, but in FIG. 5, the local volume fraction of particles 194 varies spatially in a somewhat continuous variation, rather than in an approximately stepwise manner. A still further possibility is that there could be identifiable regions such as regions 162 and 164, such that within an individual region the concentration of particles is substantially constant, but in the immediate vicinity of where the two regions meet each other, there could be a gradient of particle concentration.

In any situation (gradient or identifiable regions or other situations), the distribution of local volume fraction of particles can be such that the local volume fraction of particles within the cement may be greater in the immediate vicinity of the bone interface than it is away from the bone interface. In general, the local particle concentration may be spatially non-uniform, and may be non-zero substantially everywhere throughout the composite. These spatial variations of particle concentration may be controlled variations that achieve desired particle concentrations in specific places. The desired particle concentrations may be chosen for reasons related to biological considerations or fracture mechanics, as described elsewhere herein.

In other embodiments, it is possible that some localized region of zero local particle concentration may exist, while, at the same time, there exists a spatially non-uniform distribution of local particle concentration in that portion of the composite which does contain particles. For example, this may occur in connection with the filling of cavities in smaller bones such as vertebrae as compared to long bones in total knee and hip joint replacements.

### Materials

The particles may be biocompatible and/or bioresorbable. Specifically, in an embodiment which contains identifiable regions such as regions 162 and 164, at least the particles 172 in region 162 (which adjoins natural bone 140) may be bioresorbable. More generally, such as in embodiments that have a gradient, at least the particles, which are in the immediate vicinity of the interface with natural bone, may be bioresorbable. In a region in which bone ingrowth is desired, such as region 162, the bioresorbability of the particles 172 in that region, may allow those particles to be replaced by natural bone for the formation of a strong interfacial bond. More interiorly in the composite, such as in region 164, the particles may also be bioresorbable, but are not required to be.

Any of the particles 172 and 174 may include one or more of the following materials: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate. The particles could contain carbon in any form appropriate for use within the human body. The particles may be either osteoconductive, osteoinductive or both. If the particles are at least osteoconductive, they have been shown by Dai, K. R., Y. K. Liu, J. B. Park, C. R. Clark, K. Nishiyama and Z. K. Zheng, “Bone-particle-impregnated bone cement: An in vivo weight bearing study,” Journal of Biomedical Materials Research, Vol. 25, 141-150, 1991 that those inter-touching particles would promote the formation and ingrowth of bone into the cement through simultaneous osteoclastic and osteoblastic activities. If the particles are osteoinductive and the exothermic excursion of cement such as PMMA were to destroy some or all of the osteoinductive properties of the osteoinductive material, then some of its osteoconductivity would still remain.

As will be appreciated by one of ordinary skill in the art, examples of osteoconductive particle types include inorganic bone particles, collagen, beta tricalcium phosphate and other forms of calcium phosphate. Examples of osteoinductive particles include osteogenic protein-1, demineralized bone matrix (DBM) and bone morphogenic protein-2. Examples of both osteoconductive and osteoinductive particles include natural bone, e.g., allogenic and autogenous bone grafts as well as collagen mineral composite grafts, e.g., collagen in combination with hydroxyapatite and tricalcium phosphate.

The particles 172 and 174, or the particles in any individual region, may be a mixture of more than one kind of particle, and may have a distribution of sizes, shapes and other properties. The particles could be of any shape. In some embodiments, the particles could even have a shape which is as elongated or non-equiaxial as a fiber. Fibers can be advantageously useful as strengthening agents in composite materials.

The particles 172 in region 162 and the particles 174 in region 164 could be substantially identical to each other in all their physical properties such as composition and geometric and dimensional properties. Alternatively, the particles 172 and 174 in the two regions 162 and 164 could differ from each other in any one or more or any combination of the following properties: composition, biocompatibility, resorbability or resorption rate, size, shape, size distribution, shape distribution, or any other property. In any individual region, the composition, size shape, and any other properties of the particles in that region may be chosen appropriately to produce a composite having mechanical and material or other properties which are desired for that individual region.

In the situation where there is a gradient of particle concentration, there could also be differences from one place to another place in any of the physical properties of the particles, as just mentioned.

The particle size or distribution of particle sizes can be varied widely, depending upon the composition of the particles and the intended clinical performance. In general, particles having a size, for example, of at least about 150 microns to no greater than about 300 microns, can be optimal for osteoconductive ingrowth of bone to the composite (see J. J. Klawitter and S. F. Hulbert “Application of Porous Ceramics for the Attachment of Load Bearing Internal Orthopedic Applications,” J. Biomed. Mater. Res. Symp., 2(1), 161-229, 1972), and; J. B. Park and R. S. Lakes “Biomaterials: An Introduction—Second Edition,” Plenum Press, 1992, pp 177-178, both of which are hereby incorporated by reference in their entireties).

In some embodiments, the bone cement may be non-resorbable or may have only a very slow rate of absorption such as taking more than about 50 years to resorb in the environment of the human or animal body. The bone cement may include polymethylmethacrylate (PMMA) cement. Alternatively, or in addition, the bone cement could include any one or more of: hydroxyethyl methacrylate (HEMA); polyalkanoate; polyetherurethane; polycarbonate urethane; polysiloxaneurethane; and polyfluoroethylene. Agents that may be included in the composition of the PMMA/particulate aggregate may include thrombin, fibrinogen, epsilon-aminocaproic acid (Amicar) or other agents to prompt local clotting at the perimeter of the cavity; particulate or soluble antibiotics to preclude infection at the procedure site; growth factors to stimulate either neovascularization or otherwise facilitate incorporation of the high concentration particulate component of the implanted material, including but not limited to endothelial growth factors such as VEGF; G-CSF, GM-CSF, or thrombopoietin; contrast material to enhance visualization of the implanted material during and after the procedure; in the case of malignant replacement or bone destruction, chemotherapeutic agents in either a soluble, gel or solid phase may be introduced including but not limited to adriamycin and cisplatin; single or multiple osteogenesis-enhancing agents may also be incorporated into the compound before, during or after introduction of the cement and bioresorbable particles.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Additionally, the skilled artisan will recognize that any of the above-described methods can be carried out using any appropriate apparatus. Further, the disclosure herein of any particular feature in connection with an embodiment can be used in all other disclosed embodiments set forth herein. Thus, it is intended that the scope of the present invention herein disclosed should not be limited by the particular disclosed embodiments described above.

What is claimed is:

1. A bone implant comprising:
a mesh structure having a collapsed configuration and an expanded configuration, the mesh structure comprising a sidewall and an interior chamber, wherein the sidewall has an open-cell matrix configuration;
a first media configured to fill the sidewall of the mesh structure and promote bone ingrowth; and
a second media configured to fill the interior chamber of the mesh structure and have a crack propagation arresting characteristic.

2. The bone implant of claim 1, wherein the mesh structure is at least partially bioresorbable.

3. The bone implant of claim 1, wherein the first media and the second media comprise particles; wherein a particulate density of the first media is greater than a particulate density of the second media.

4. The bone implant of claim 1, wherein the first media includes particles in a concentration within the range of from about 50% to about 80% by weight.

5. The bone implant of claim 1, wherein the second media includes particles within the range of from about 10% to about 50% by weight.

6. The bone implant of claim 1, wherein the second media includes particles within the range of from about 25% to about 35% by weight.

7. The bone implant of claim 1, wherein the first media comprises particles having a size within the range of from about 50 microns to about 500 microns.

8. The bone implant of claim 1, wherein the first media comprises particles having a size within the range of from about 150 microns to about 300 microns.

9. The bone implant of claim 1, wherein at least one of the first and second media comprises PMMA.

10. The bone implant of claim 1, wherein the sidewall of the bone implant comprises pores having a size of between about 0.5 mm and 1 mm.

11. The bone implant of claim 1, wherein the mesh structure has a diameter of between about 1 mm and 4 mm in its expanded configuration.

12. A kit for performing vertebroplasty, comprising:
a bone implant comprising a mesh structure having a collapsed configuration and an expanded configuration, the mesh structure comprising a sidewall and an interior chamber, wherein the sidewall has an open-cell matrix configuration;
a first media configured to fill the sidewall of the mesh structure and promote bone ingrowth;
a second media configured to fill the interior chamber of the mesh structure and have a crack propagation arresting characteristic;
a vertebroplasty catheter comprising a proximal end, a distal, end, and an elongate tubular body, the tubular body having a central lumen extending therethrough, wherein the mesh structure and the vertebroplasty catheter are configured to be releasably coupled together.

13. The kit for performing vertebroplasty of claim 12, wherein the mesh structure and the vertebroplasty catheter comprise complementary threaded attachment structures.

14. A method for treating the spine, comprising the steps of:
inserting an insertion device percutaneously into a vertebral body;
introducing a bone implant comprising a mesh structure having a collapsed configuration and an expanded configuration, the mesh structure comprising a sidewall and an interior chamber, wherein the sidewall has an open-cell matrix configuration;
filling the sidewall of the mesh structure with a first media having a bone ingrowth characteristic;
filling the interior chamber of the mesh structure with a second media having a crack propagation arresting characteristic.

15. The method of claim 14, further comprising inserting a cavity-forming device through the insertion device into an area of cancellous bone in the vertebral body; and
displacing cancellous bone with the cavity-forming device to create a cavity defined by a surface of cancellous bone.

16. A method for treating the spine, comprising the steps of:
inserting a deployment device into a vertebral body, the deployment device releasably carrying an inflatable container having a central cavity and a porous sidewall;
inflating the container within the vertebral body;
releasing the container within the vertebral body; and
removing the deployment device from the vertebral body;
wherein the released container comprises a first media within the pores of the sidewall and a second media within the cavity.

17. A method as in claim 16, wherein the first media is introduced into the pores prior to the inserting step.

18. A method as in claim 16, wherein the first media is introduced into the pores following the inserting step.

19. A method as in claim 16, wherein the inflating step compacts adjacent cancellous bone.

20. A method as in claim 16, further comprising the step of creating a cavity within the vertebral body prior to the inserting step.

21. A method as in claim 16, wherein the pores comprise spaces between fibers.

22. A method as in claim 16, wherein the pores comprise open, interconnected cells in a porous matrix.

23. A method as in claim 16, wherein the inserting step is accomplished through an insertion cannula.
